(19) United States
(12) Patent Application Publication
(10) Pub. No.: US 2012/0083804 A1
(43) Pub. Date: Apr. 5, 2012

(54) MARKER CLIP DEVICE

(75) Inventor: Gregory J. Skerven, Kernersville, NC (US)

(73) Assignee: Cook Medical Technologies LLC, Bloomington, IN (US)

(21) Appl. No.: 13/080,925

(22) Filed: Apr. 6, 2011

Related U.S. Application Data
(60) Provisional application No. 61/321,953, filed on Apr. 8, 2010.

(51) Int. Cl. A61B 17/10

(52) U.S. Cl. 606/142

(57) ABSTRACT

A marker clip device and system for marking tissue within a body cavity comprising a plurality of deployable marker clips having unique markings or other indicia to thereby allow each clip to be individually identified subsequent to clip deployment. A clip delivery device configured to deploy the plurality of marker clips at various locations within a body cavity is also provided.

### CROSS-REFERENCE TO RELATED APPLICATIONS

The present application claims the benefit of U.S. Provisional Application No. 61/321,953, titled “Marker Clip Device”, filed Apr. 8, 2010, the entirety of which is hereby incorporated by reference.

### TECHNICAL FIELD

The present invention relates to a marker clip device, and more specifically to a device and system for delivering one or more marker clips to an internal cavity or lumen of a mammalian body, such as the gastrointestinal tract of a human patient. The device and system may be use to mark or hold tissue or the like, or may be used to cause hemostasis of blood vessels within the body cavity of the patient.

### BACKGROUND OF THE INVENTION

Conventionally, a clip may be introduced into a body cavity through an endoscope to grasp living tissue of a body cavity for hemostasis, marking, and/or ligating. In addition, clips are now being used in a number of applications related to gastrointestinal bleeding such as peptic ulcers, Mallory-Weiss tears, Dieulafoy's lesions, angiomas, post-papillotomy bleeding, and small varices with active bleeding.

One example of a clip device that is used primarily for hemostasis is described in U.S. Pat. No. 7,122,041, titled “Clip Device”, which discloses a clip having at least three arms that may be closed onto tissue. The clip device includes an outer sheath that is insertable into a body cavity, an inner sheath that is slidably disposed within the outer sheath, and an operating wire that is movably disposed through the inner sheath. A retainer is attached to the distal portion of the operating wire and is configured to mate with a retainer on the proximal end of the clip. A sliding ring is movably about the arms of the clip and is configured to prevent the retainers from separating while disposed thereabout. The clip is deployed by moving the inner sheath distally relative to the operating wire so as to engage and move the sliding ring in a distal direction along the arms of the clip. The distal movement of the sliding ring closes the arms of the clip and simultaneously exposes the retainers, thereby allowing the retainers to separate to release the clip from the operating wire.

Another example of a clip device is described in U.S. Patent Publication No. 2006/0224165, titled “Multi-Clip device”, which discloses a device for delivering a plurality of clips. The device includes a sheath that is insertable into a body cavity, and an operating wire that is movably disposed through the sheath. A plurality of clips are disposed within the sheath and are operably connected to the operating wire in a head to tail fashion, i.e., the arms of each clip are connected to the adjacent distal clip. Each clip includes two or more arms that are biased in the open position, and an expandable collar that is slidably disposed about the arms of the clip. The clips are serially deployed by withdrawing the sheath in a proximal direction so as to expose the collar of the distal-most clip, thereby allowing the collar to expand in diameter. The sheath is then advanced distally to engage and push the expanded collar along the arms of the clip, thereby closing the clip. The deployed clip is released by retracting the sheath until the arms of the next clip open sufficiently to disengage from the deployed clip.

The above-described clip devices are used primarily for causing hemostasis of blood vessels within the body cavity of a patient. These devices may, however, also be used to mark tissue within the body cavity. For example, the physician may want to mark tissue for treatment by a subsequent procedure such as sampling, resection, cauterization, or band ligation. In particular, the physician may use the above-described clip devices to attach one or more clips to tissue at various locations within the body cavity. The delivery portion of the clip device is removed once the clips have been deployed at the desired target locations. The deployed clips may then be used to guide one or more medical devices configured for performing the desired medical procedures to the target locations within the body cavity.

One disadvantage of using the above-described clip devices to mark tissue is that the clips are generally of identical design for each type of device. Thus, if the physician uses more than one clip to mark tissue at various locations for subsequent treatment by different medical procedures, it may be difficult or impossible to determine which clip has been used to mark a particular tissue. This is because it is generally desirable to mark all tissues for subsequent treatment or monitoring at one time so as to expedite the entire procedure and reduce trauma to the patient. For example, the physician may mark a first tissue that requires sampling, a second tissue that requires resection, and a third tissue for monitoring. However, once the tissues have been marked and the clip device removed, it may be difficult to distinguish the three marked tissues, particularly if a different physician is performing the subsequent medical procedure. The problem may be compounded if the subsequent medical procedure is delayed for a period of time after the initial marking. Such a delay is typical if tissue has been marked for monitoring over an extended period of time.

Using identical clips to mark tissue at more than one location within the body cavity is also a problem when marking multiple tissues for sampling. For example, the physician may use one of the above-described clip devices to mark multiple tissues to be sampled during a subsequent biopsy procedure. However, once the samples have been excised from the surrounding tissue and removed from the patient, it may be difficult or impossible to determine which sample was excised from a particular biopsy site. As a result, any sample that indicates that additional treatment or examination is warranted cannot be matched up with the biopsy site. Even careful documentation and control of the individual samples may not be sufficient to avoid confusion or mixing of the samples.

The marker clip device of the present invention solves the above and other problems by providing marker clips that are individually identifiable and/or distinguishable.

### SUMMARY OF THE INVENTION

A marker clip device and system for marking tissue within a body cavity according to the present invention comprises a plurality of deployable marker clips having unique markings or other indicia to thereby allow each clip to be individually identified subsequent to clip deployment.

In one aspect, the present invention comprises a plurality of deployable marker clips that are individually color coded. Specifically, each of the plurality of clips may be coated or formed of a material having a different color. For example, the plurality of marker clips may comprise a set of four marker clips, wherein each of the marker clips comprises, respectively, a red, blue, green or black colored coating.

In another aspect, the present invention comprises a plurality of deployable marker clips that are individually marked with alpha-numeric symbols. Specifically, each of the plurality of marker clips may be embossed or etched with a different number or letter. For example, the plurality of marker clips may comprise a set of five marker clips, wherein each of the marker clips has been etched, respectively, with the numerals 1, 2, 3, 4 or 5.

In another aspect, the present invention comprises a kit including a plurality of deployable marker clips having unique markings or other indicia. The kit further comprises a clip delivery device for delivering the plurality of marker clips. The clip delivery device may be configured to deliver either a single marker clip or multiple marker clips. The kit may also include packaging to contain and maintain the delivery device and marker clips in an organized and sterilized condition prior to use.

In another aspect, the present invention comprises a method for marking a plurality of tissues at various locations within the body cavity. The method includes the steps of marking a plurality of target tissues with individually deployable marker clips, wherein each marker clip has a unique marking or other indicia. The method may further include the step of performing a therapeutic or diagnostic medical procedure to one or more of the target tissues identified by a deployed marker clip.

### BRIEF DESCRIPTION OF THE DRAWINGS

FIG. 1 is an illustration of one embodiment of the marker clip device according to the present invention.

FIG. 2 is an illustration of the distal portion of the marker clip device of FIG. 1 before deployment of a marker clip.

FIG. 3 is an illustration of the portion of the marker clip device of FIG. 1 during a first stage of deployment of a marker clip.

FIG. 4 is an illustration of the portion of the marker clip device of FIG. 1 during a second stage of deployment of a marker clip.

FIG. 5 is an illustration of the portion of the marker clip device of FIG. 1 after deployment and release of a marker clip.

FIG. 6 is an illustration of a first embodiment of the clip portion of the marker clip of the present invention.

FIG. 7 is an illustration of a second embodiment of the clip portion of the marker clip of the present invention.

FIG. 8 is an illustration of an embodiment of the expandable collar portion of the marker clip of the present invention.

FIG. 9 is an illustration of a set of deployable marker clips that are individually color coded.

FIG. 10 is an illustration of a set of deployable marker clips that individually marked with alpha-numeric symbols.

### DESCRIPTION OF THE INVENTION

The present invention provides a marker clip device and system for marking tissue within a body cavity. Specifically, the present invention comprises a plurality of deployable marker clips having unique markings or other indicia to thereby allow each clip to be individually identified subsequent to clip deployment.

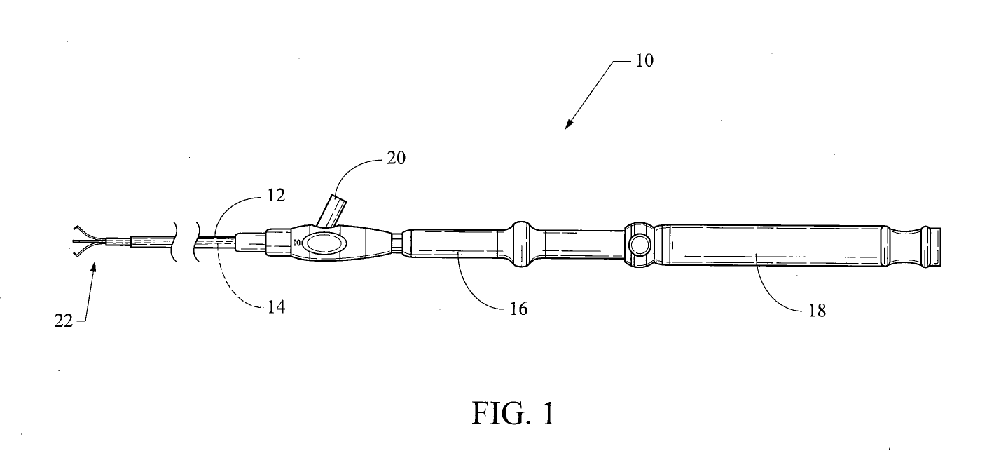

A first embodiment of the present invention is shown in FIGS. 1-9, which illustrates a marker clip device for delivering a plurality of uniquely colored marker clips. Referring to FIG. 1, a clip delivery device according to the present invention is shown. The marker clip device 10 includes an introducing tube 12 that is insertable into the body cavity. Disposed within the introducing tube (also referred to as the sheath) is an operating wire 14 (also referred to as a drive cable). The operating wire 14 is independently slidable within the introducing tube 12. In other words, the operating wire 14 can be advanced and retracted independently of the movement of the introducing tube 12.

The introducing tube 12 is attached at its proximal end to a forward handle portion 16. The operating wire 14 extends through the forward handle portion 16 and is attached at its proximal end to a rearward handle portion 18, which is disposed proximally of the forward handle portion 16. The rearward handle portion 18 telescopically extends over the proximal portion of the forward handle portion 16. As will be explained in more detail below, longitudinal movement of the operating wire 14 relative to the introducing tube 12 is controlled by longitudinal manipulation of the forward handle portion 16 relative to the rearward handles portion 18.

The forward handle portion 16 also includes a flushing port 20. The flushing port 20 can comprise a standard male or female luer fitting, or any other valve mechanism that permits the injection of fluid there through. The flushing port 20 is in fluid communication with the interior volume of the forward handle portion 16, which in turn is in fluid communication with the cavity or lumen within the introducing tube 12. Accordingly, any fluid injected through the flushing port 20 will necessarily enter the cavity or lumen of the introducing tube 12, and will subsequently exit the cavity near the distal end of the introducing tube 12. In other words, the fluid injected through the flushing port 20 will exit the distal end of the marker clip device 10.

Alternatively, the cavity or lumen can be disposed within the wall of the introducing tube 12. In other words, the introducing sheath can comprise a separate lumen disposed through which fluid can be passed along the length thereof. It should also be understood that the flushing port 20 could be alternatively located on the middle rearward handle portion 18, or on a portion of the introducing tube 12 distally of any of the handle portions.

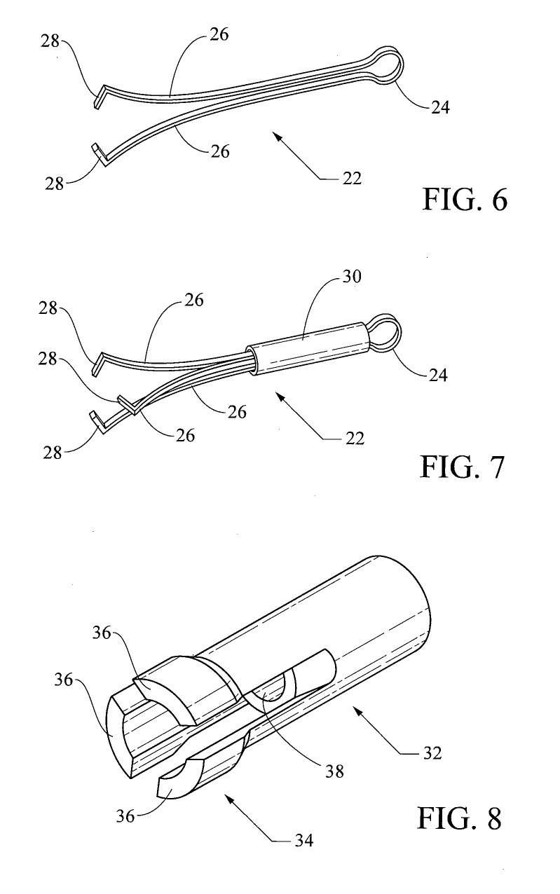

The marker clip device 10 further includes a plurality of detachable and deployable marker clips 22 disposed within the introducing tub 12. As illustrated in FIGS. 6 and 7, each marker clip 22 has an engagement portion 24 at the proximal end thereof and a plurality of arms 26 extending towards the distal end thereof. The arms 26 are formed of a resilient material and are shaped such that the arms 26 have a tendency to be in an open position towards the distal end of the clip 22. Each arm 26 includes an inwardly facing tooth 28 which is configured to grasp the target tissue. The teeth 28 preferably overlap with each other when the marker clip 22 is in the closed position. As will be explained in greater detail below, the engagement portion 24 is configured such that it can be engaged by the distal end of the operating wire 14 or by the closed arms 26 of another clip 22.

In the embodiment illustrated in FIG. 6, the marker clip 22 comprises a pair of arms 26 formed by bending a single elongate piece of resilient material. The engagement portion 24 is likewise formed by bending the central portion of the elongate piece of resilient material to form a loop.

In the embodiment illustrated in FIG. 7, the marker clip 22 comprises three arms 26. Each arm 26 is individually formed from a resilient material and affixed to a central tube 30 by any suitable means such as welding or gluing. The engagement portion 24 is likewise individually formed and attached to a proximal end of the central tube 30. The use of three arms allows the marker clip 22 to grasp the target tissue with minimal, if any, need to rotate the marker clip 22 into the correct orientation. While three arms are illustrated in this embodiment, it is contemplated that more than three arms may be used.

The marker clip 22 may be made from any suitable resilient material such as stainless steel, nitinol, plastic, and the like, and is preferably a biocompatible material. The material used for the marker clip 22 may also be bio-degradable. In addition, the arms may have a cross-sectional shape that is round, square, triangular, pie-shaped, truncated cone, and the like. A triangular or delta shaped cross-section is particularly advantageous for a clip having three arms because it allows a reduction in the cross-sectional area that the arms occupy within the introducing tube 12, thereby allowing a reduction in the diameter of the introducing tube 12.

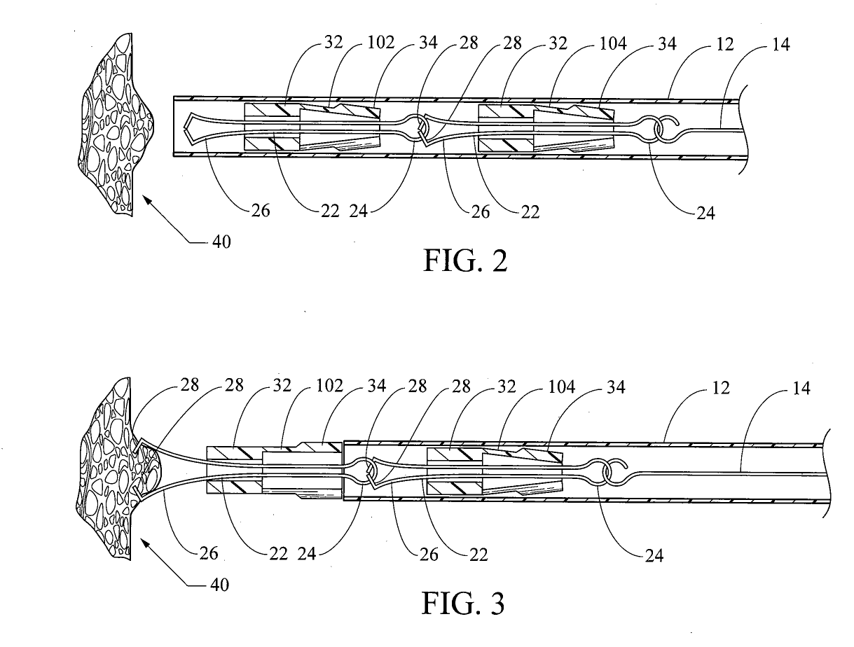

An expandable collar 32 is slidably disposed about the arms 26 of the marker clip 22 and is configured such that when the collar 32 is moved distally over the arms 26 it closes and holds them in a closed position. As illustrated in FIG. 8, the collar 32 has an expanding portion 34 that is movable from a first compressed position to second expanded position. When in the first compress position, the collar 32 is slidably disposed within the introducing tube 12 (FIG. 2), and when in the second expanded position (FIG. 3), the collar 32 may be engaged by the distal end of the introducing tube 12 so that the introducing tube 12 can be used to advance the collar 32 distally relative to the marker clip 22.

In the embodiment illustrated in FIG. 8, the expanding portion 34 comprises a plurality of movable arms 36 extending towards the proximal end of the collar 32. The movable arms 36 are biased in an outward transverse direction so as to have an outer diameter that is greater than the inside diameter of the introducing tube 12. However, and as will be explained in greater detail below, the movable arms 36 are compressible to allow the collar 32 to slidably fit within the introducing tube 12. In the alternative, the expanding portion 34 may comprise a resilient material, such as rubber, that is compressible from the expanded position to the compressed position. Such an embodiment would not require any movable components.

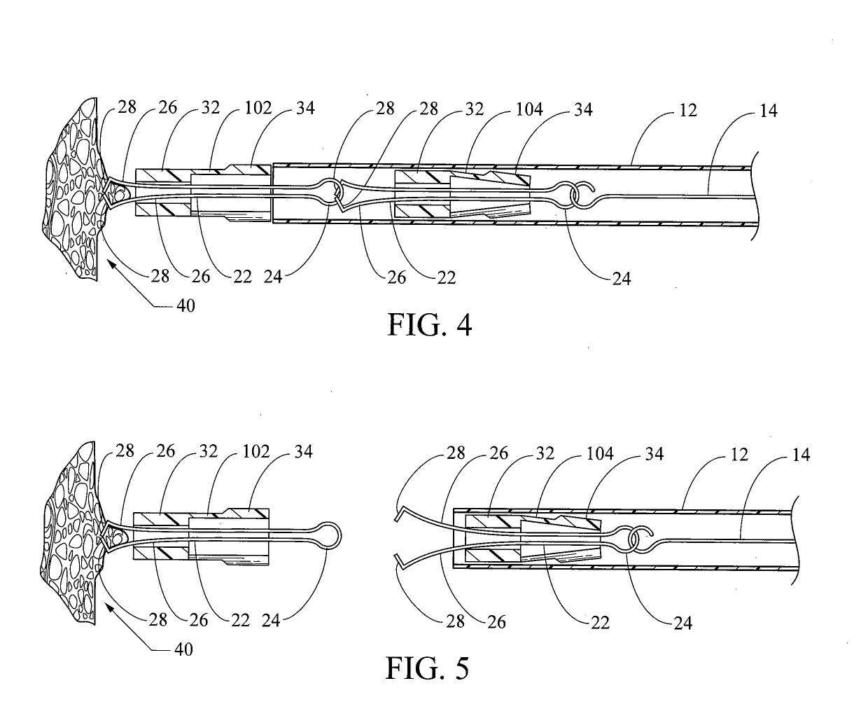

The collar 32 further comprises a central lumen 38 through which the arms 26 of the marker clip 22 are slidably disposed. The central lumen 38 is sized so as to close the arms 26 of the marker clip 22 as the collar 32 is advanced towards the distal end of the marker clip 22 (see FIGS. 4 and 5). In other words, the wall portion of the collar 32 that defines the central lumen 38 is configured to engage and overcome the transverse outwardly directed biasing force of the arms 26 so as to push the teeth 28 of the arms 26 into an overlapping arrangement as the collar 32 is moved distally relative to the marker clip 22. The central lumen 38 may also be sized so as to prevent the proximal end of the marker clip 22, i.e., the engagement portion 24, from passing there through so as to prevent the collar 32 from separating from the marker clip 22.

The exterior surface of the collar 32 may also be sized and/or configured to allow the passage of fluids around or through the collar 32. As will be explained below, it may be desirable, for example, to pass saline through the introducing tube 12 to flush any blood or bodily fluids away from the part to be treated. Thus, the collar 26 may be sized to provide a gap between the exterior surface of the collar 26 and the interior surface of the introducing tube 12 through which fluids can pass. Alternatively, the collar 26 may include a flow channel or lumen extending there through.

The expandable collar 32 may be made from any suitable resilient material such as plastic, rubber, stainless steel, nitinol, and the like, and is preferably a biocompatible material. The material used for the collar 32 may also be bio-degradable. The expandable collar 32 can be manufactured by any suitable procedure, such as milling (in the case of metal materials) or injection molding (in the case of plastic and rubber materials). While three movable arms 36 are illustrated in the embodiment shown in FIG. 8, it is contemplated any number of movable arms or components may be used. For example, the collar 32 may comprise a single arm or other type of movable or expandable device that can extend outwardly in a transverse direction so as to be engaged by the distal end of the introducing tube 12.

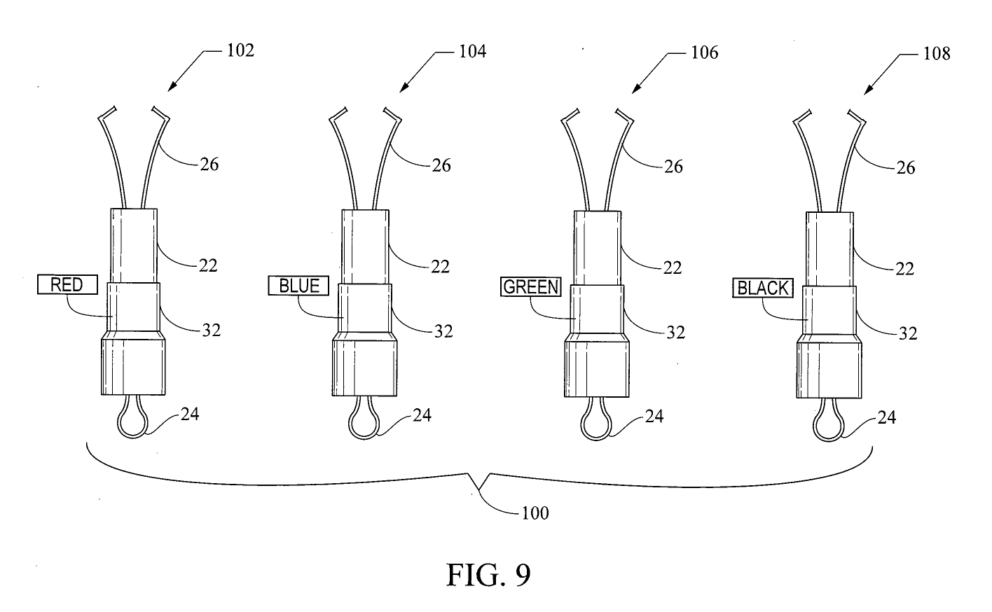

FIG. 9 illustrates a plurality of deployable marker clips 22 that are individually color coded so as to form a set 100 of marker clips 22. Specifically, each of the plurality of marker clips 22 is coated or formed of a material having a different color. For example, and as illustrated in FIG. 9, the set 100 of marker clips 22 comprises a set of four marker clips 102, 104, 106 and 108, respectively. Each of the marker clips 102, 104, 106 and 108 comprises a collar 32 having a unique color. Specifically, marker clip 102 comprises a collar 32 that is red colored, marker clip 104 comprises a collar 32 that is blue colored, marker clip 106 comprises a collar 32 that is green colored, and marker clip 108 comprises a collar 32 that is black colored. The arms 26 and/or engagement portions 24 of the marker clips 22 may likewise (or alternatively to the collars 32) comprise a unique color. In the particular embodiment illustrated, the individual collars 32 for each of the marker clips 102, 104, 106 and 108 are molded from differently colored plastic materials. Alternatively, the individual collars 32 may be molded or formed from the same material and subsequently coated with different colors.

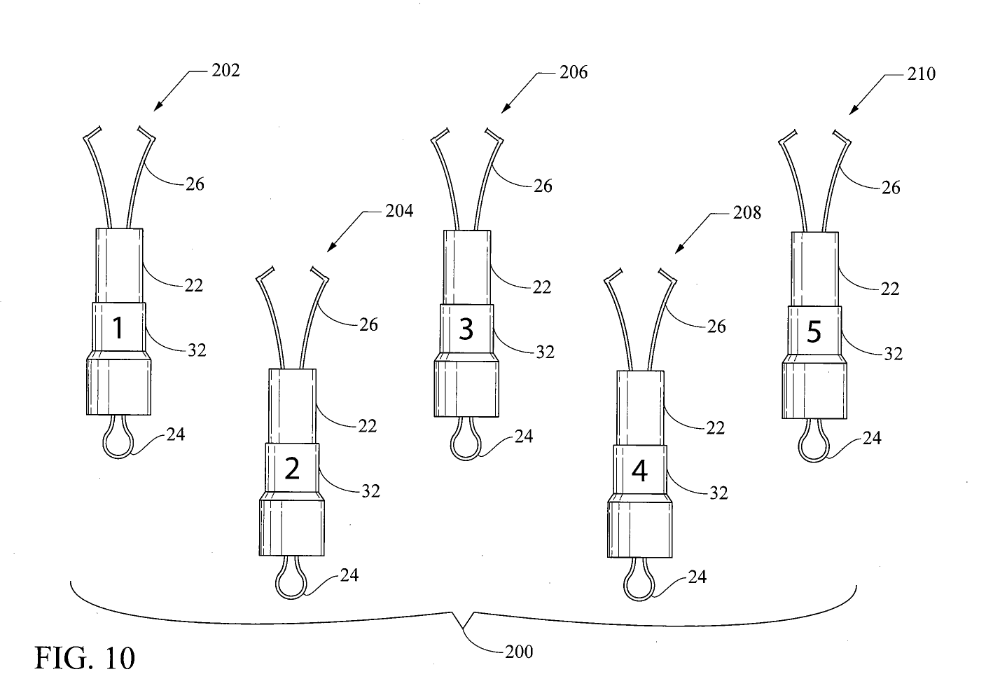

FIG. 10 illustrates a plurality of deployable marker clips 22 that are individually marked with an alpha-numeric symbol so as to form a set 200 of marker clips 22. Specifically, each of the plurality of marker clips 22 is etched or embossed with a different number or letter. For example, and as illustrated in FIG. 10, the set 200 of marker clips 22 comprises a set of five marker clips 202, 204, 206, 208 and 210, respectively. Each of the marker clips 202, 204, 206, 208 and 210 comprises a collar 32 marked with a unique color alpha-numeric symbol. Specifically, marker clip 202 comprises a collar 32 that is marked with the numeral “1”, marker clip 204 comprises a collar 32 that is marked with the numeral “2”, marker clip 206 comprises a collar 32 that is marked with the numeral “3”, marker clip 208 comprises a collar 32 that is marked with the numeral “4”, and marker clip 210 comprises a collar that is marked with the numeral “5”. Preferably, the alpha-numeric symbols are marked on at least two opposing exterior sides of the collar 32 so as to be visible from multiple directions. In the embodiment illustrated, the alpha-numeric symbols are formed by laser etching the exterior surface of collar, although other methods of forming the symbols are contemplated.

A plurality of deployable marker clips 22 having unique markings or other indicia, such as the sets 100, 200 illustrated in FIGS. 9 and 10, respectively, are preferably supplied or packaged in the form of a kit that also includes the delivery device. The kit may also include packaging to contain and maintain the delivery device and marker clips 22 in an organized and sterilized condition prior to use.

As best seen in FIG. 2, a plurality of marker clips 22 are arranged inside the introducing tube 12 in a serial fashion. The distal end of the operating wire 14 engages the engagement portion 24 of the proximal most marker clip 22 (i.e., marker clip 104). The arms 26, and more specifically the teeth 28, of marker clip 104 engage the engagement portion 24 of the adjacent distally located clip 22 (i.e., marker clip 102). The inside surface of the introducing tube 12 maintains the arms 26 in a closed position through or about the engagement portion 24 of the adjacent clip. The expandable collar 32 helps to maintain the position of each clip 22 within the introducing tube 12.

In the particular embodiment illustrated, two uniquely identifiable marker clips 102, 104 are disposed inside the introducing tube 12. As explained above in connection with the discussion of FIG. 9, marker clip 102 comprises a collar 32 that is red colored and marker clip 104 comprises a collar that is blue colored. Although only two marker clips 102, 104 are shown in FIG. 2, it should be understood that any number or combination of uniquely identifiable marker clips (such as the marker clips 22 of the sets 100, 200 shown in FIGS. 9 and 10) may be arranged inside the introducing tube 12. Additional marker clips may be connected to each other in the same “head-to-tail” fashion as shown in FIG. 2. In other words, each marker clip 22 is connected to the operating 14 wire via each of the proximally located intervening clips. Thus, each intervening clip 22 functions as an extension of the operating wire 14. This arrangement eliminates the need for separate operating wires connected to each of individual marker clips.

The operation of the marker clip device 10 will be described. Prior to introduction of the device 10 into the patient, a plurality of uniquely identifiable marker clips 22 (i.e., marker clips 102, 104) are disposed in the introducing tube 12 and connected to the operating wire 14 as shown in FIG. 2. This may be accomplished by connecting the engagement portion 24 of marker clip 104 to the operating wire 14, and then retracting the operating wire 14 so as to draw the marker clip 104 into the introducing tube 12. The expandable collar 32 of marker clip 104 must be compressed so as to allow the collar 32 to be drawn into the introducing tube 12. The engagement portion 24 of a second marker clip 22 (i.e., marker clip 102) is then connected to the arms 26 of marker clip 104. The operating wire 14 is then further retracted so as to draw marker clip 102, along with its expandable collar 32, into the introducing tube 12. This procedure is repeated until the desired number of marker clips 22 are loaded into the introducing tube 12 of the device 10.

The introducing tube 12 is then introduced into a body cavity via a channel of an endoscope or similar device that has been previously inserted into the body cavity. While the body cavity is observed via the endoscope, the distal end portion of the outer introducing tube 12 is guided to the part to be treated.

If the part to be treated is obscured by blood or other bodily fluids, then a fluid such as saline is injected through the flushing port 20 on the forward handle portion 16. The saline enters the cavity or lumen within the introducing tube 12 and exits the distal end thereof. The saline floods the area so as to flush any blood or bodily fluids away from the part to be treated. The injection of saline is continued and/or repeated as necessary during the following steps so as to keep the area free of blood and other bodily fluids.

Alternatively, a vacuum is applied to the flushing port 20 so as to create suction within the cavity or lumen within introducing tube 12. This suction can be used to remove blood or other bodily fluids from the area surrounding the part to be treated.

To deploy the marker clip 22, the operating wire 14 is advanced distally relative to the introducing tube 12 to extend the arms 26 of the distal most marker clip 22 (i.e., marker clip 102) out of the distal end of the introducing tube 12. As shown in FIG. 3, the distal most marker clip 22, 102 is advanced far enough to allow the expandable portion 34 of the collar 32 to expand to its second configuration. The extended marker clip 22, 102 can now be positioned about the target tissue.

Once the arms 26 of the marker clip 22, 102 are positioned about the target tissue, the operating wire 14 is retracted in a proximal direction (or the introducing tube 12 is advanced in a distal direction) to engage the collar 32 with the distal end of the introducing tube 12. More specifically, the proximal movement of the operating wire 14 relative to the introducing tube 12 causes the distal end of the introducing tube 12 to engage the expanding portion 34 of the collar 32. As shown in FIG. 4, further proximal movement of the operating wire 14 relative to the introducing tube 12 causes the collar 32 to slide over the arms 26 of the marker clip 22, 102 so as to close the arms 26 onto the tissue. In other words, as the marker clip 22, 102 is being pulled or drawn proximally into the introducing tube 12, the expanding portion 34 prevents the collar 32 from being likewise pulled or drawn into the introducing tube 12. The result is that the collar 32 is pushed distally relative to the marker clip 22, 102.

Once the marker clip 22, 102 is secured to the target tissue (i.e., deployed), the operating wire 14 is advanced in a distal direction (or the introducing tube 12 is retracted in a proximal direction) to release or detach the deployed marker clip 22, 102 from the delivery device 10. More specifically, and as shown in FIG. 5, the operating wire 14 is advanced in a distal direction relative to the introducing tube 12 so as to push the next proximally located marker clip 22 (i.e., marker clip 104) and collar 32 assembly towards the distal end of the introducing tube 12. Once the arms 26 of the next proximally located marker clip 22, 104 have been extended out of the distal end of the introducing tube 12 a distance sufficient to open the arms 26, the arms 26 disengage from the engagement portion 24 of the deployed marker clip 22, 102 to thereby release the deployed marker clip 22, 102 from the delivery device 10. The delivery device 10 can then be used to deploy the next marker clip 22, 104 by following the same procedure described above.

The above-described device and procedure allows for the individual marking of target tissue at various locations within the body cavity. Moreover, the device and procedure allows the individually marked tissues to be separately identified and distinguished from other marked tissues, even if the marked tissues are subsequently removed for diagnostic or therapeutic medical procedures.

It should also be understood that any number of uniquely identifiable marker clips 22 can be initially loaded into the introducing tube 12. Each of these marker clips 22 would then be available for deployment during the medical procedure without the need to withdraw the marker clip device 10 for re-loading after each deployment, and without the need to insert additional clip delivery devices.

In an alternative embodiment of the present invention, the marker clip device may comprise a clip delivery device configured to deliver only one marker clip at a time. Such a clip delivery device may comprise the type of clip and delivery device disclosed in U.S. Pat. No. 7,122,041, titled “Clip Device”, the entire contents of which are hereby incorporated by reference. Specifically, the marker clip device may comprise an outer sheath that is insertable into a body cavity, an inner sheath that is slidably disposed within the outer sheath, and an operating wire that is movably disposed through the inner sheath. A retainer may be attached to the distal portion of the operating wire and configured to mate with a retainer on the proximal end of the marker clip. A sliding ring may be movably disposed about the arms of the marker clip and configured to prevent the retainers from separating while disposed thereabout. The marker clip may be deployed by moving the inner sheath distally relative to the operating wire so as to engage and move the sliding ring in a distal direction along the arms of the marker clip. The distal movement of the sliding ring closes the arms of the marker clip and simultaneously exposes the retainers, thereby allowing the retainers to separate to release the marker clip from the operating wire.

A plurality of uniquely identifiable marker clips, each having the same functional structure of the above-described marker clip, would be provided with the marker clip device. These components would preferably be supplied or packaged in the form of a kit. The kit would enable a user to use the marker clip device to mark multiple target tissues with uniquely identifiable marker clips.

While there have been described what are presently believed to be the preferred embodiments of the invention, those skilled in the art will realize that changes and modifications may be made thereto without departing from the spirit of the invention. It is to be understood that the invention can be carried out by specifically different equipment and devices, and that various modifications, both as to the equipment details and operating procedures, can be accomplished without departing from the scope of the invention itself.

1. A marker clip device comprising:
a) a plurality of individually identifiable marker clips; and
b) a clip delivery device configure to deploy each of the plurality of individually identifiable marker clip at a separate target location within a body cavity.

2. The marker clip device according to claim 1, wherein the clip delivery device comprises an introducing tube and an operating wire disposed within the introducing tube, the operating wire being advanceable and retractable relative to the introducing tube.

3. The marker clip device according to claim 2, wherein the plurality of individually identifiable marker clips each comprise an engagement portion, a plurality of arms, and a slidable collar disposed about the plurality of arms, said slidable collar being movable between a first position when said marker clip is in the open position and a second position when said marker clip is in the closed position, said slidable collar being configured to cause a distal end of the plurality arms to be move towards each other when in the second position, the slidable collar comprising an expandable portion being movable between a compressed position when said marker clip is disposed within the introducing tube and an expanded position when said marker clip is at least partially disposed beyond a distal end of the introducing tube, said expandable portion being configured to be engaged by the distal end of the introducing tube when in the expanded position.

4. The marker clip device according to claim 3, wherein the plurality of individually identifiable marker clips are serially disposed within the introducing tube, and wherein the operating wire is connected to the engagement portion of the proximal most marker clip, and further wherein the plurality of arms of the proximal most marker clip are connected to the engagement portion of the distally located adjacent marker clip.

5. The marker clip device according to claim 3, wherein the expandable portion comprises a plurality of movable arms biased in and transverse outward direction, said movable arms having diameter that is equal or less than an interior diameter of the introducing tube when in the compressed position, and having a diameter that is greater than the interior diameter of the introducing tube when in the expanded position.

6. The marker clip device according to claim 1, wherein the plurality of individually identifiable marker clips comprises a set of marker clips each having a different color.

7. The marker clip device according to claim 1, wherein the plurality of individually identifiable marker clips comprises a set of marker clips each having unique and separately distinguishable alpha-numeric symbol.
